(19) United States
(12) Patent Application Publication
(10) Pub. No.: US 2024/0003820 A1
(43) Pub. Date: Jan. 4, 2024

(54) SURGICAL SYSTEM AND METHODS FOR INSTRUMENT ASSESSMENT AND CLEANING

(71) Applicant: Cilag GmbH International, Zug (CH)

(72) Inventors: Frederick E. Shelton, IV, Hillsboro, OH (US); Jacqueline C. Aronhalt, Loveland, OH (US); Matthew E. Derrico, Milford, OH (US); Cameron D. McLain, Deer Park, OH (US); Bruce Brunson, Cincinnati, OH (US); Gregory A. Trees, Loveland, OH (US)

(21) Appl. No.: 17/854,127

(22) Filed: Jun. 30, 2022

(51) Int. Cl. G01N 21/88; A61B 34/20; A61B 90/70; B25J 15/00; G01N 21/31

(52) U.S. Cl. CPC G01N 21/88; A61B 34/20; A61B 90/70; B25J 15/0019; G01N 21/31

(57) ABSTRACT

A method of determining a recovery capacity of at least one feature of a surgical instrument includes establishing communication with the surgical instrument; assisting operation of the surgical instrument during a procedure; obtaining data related to the surgical instrument; evaluating the data obtained related to the surgical instrument to determine a digital assessment of the impact on performance of the at least one features of the surgical instrument; and determining, based on the digital assessment, a capacity of recovery for the at least one feature of the surgical instrument.

### BACKGROUND

A variety of ultrasonic surgical instruments include an end effector having a blade element that vibrates at ultrasonic frequencies to cut and/or seal tissue (e.g., by denaturing proteins in tissue cells). These instruments include one or more piezoelectric elements that convert electrical power into ultrasonic vibrations, which are communicated along an acoustic waveguide to the blade element. Examples of ultrasonic surgical instruments and related concepts are disclosed in U.S. Pub. No. 2006/0079874, entitled “Tissue Pad for Use with an Ultrasonic Surgical Instrument,” published Apr. 13, 2006, now abandoned, the disclosure of which is incorporated by reference herein, in its entirety; U.S. Pub. No. 2007/0191713, entitled “Ultrasonic Device for Cutting and Coagulating,” published Aug. 16, 2007, now abandoned, the disclosure of which is incorporated by reference herein, in its entirety; and U.S. Pub. No. 2008/0200940, entitled “Ultrasonic Device for Cutting and Coagulating,” published Aug. 21, 2008, now abandoned, the disclosure of which is incorporated by reference herein, in its entirety.

Some instruments are operable to seal tissue by applying radiofrequency (RF) electrosurgical energy to the tissue. Examples of such devices and related concepts are disclosed in U.S. Pat. No. 7,354,440, entitled “Electrosurgical Instrument and Method of Use,” issued Apr. 8, 2008, the disclosure of which is incorporated by reference herein, in its entirety; U.S. Pat. No. 7,381,209, entitled “Electrosurgical Instrument,” issued Jun. 3, 2008, the disclosure of which is incorporated by reference herein, in its entirety.

Some instruments are capable of applying both ultrasonic energy and RF electrosurgical energy to tissue. Examples of such instruments are described in U.S. Pat. No. 9,949,785, entitled “Ultrasonic Surgical Instrument with Electrosurgical Feature,” issued Apr. 24, 2018, the disclosure of which is incorporated by reference herein, in its entirety; U.S. Pat. No. 8,663,220, entitled “Ultrasonic Electrosurgical Instruments,” issued Mar. 4, 2014, the disclosure of which is incorporated by reference herein, in its entirety; U.S. Pat. No. 10,835,307, entitled “Modular Battery Powered Handheld Surgical Instrument Containing Elongated Multi-Layered Shaft,” issued Nov. 17, 2020, the disclosure of which is incorporated by reference herein, in its entirety; and U.S. Pat. No. 11,229,471, entitled “Modular Battery Powered Handheld Surgical Instrument with Selective Application of Energy Based on Tissue Characterization,” issued Jan. 5, 2022, the disclosure of which is incorporated by reference herein, in its entirety.

In some scenarios, it may be preferable to have surgical instruments grasped and manipulated directly by the hand or hands of one or more human operators. In addition, or as an alternative, it may be preferable to have surgical instruments controlled via a robotic surgical system. Examples of robotic surgical systems and associated instrumentation are disclosed in U.S. Pat. No. 10,624,709, entitled “Robotic Surgical Tool with Manual Release Lever,” published on May 2, 2019, the disclosure of which is incorporated by reference herein, in its entirety; U.S. Pat. No. 9,314,308, entitled “Robotic Ultrasonic Surgical Device With Articulating End Effector,” issued on Apr. 19, 2016, the disclosure of which is incorporated by reference herein, in its entirety; U.S. Pat. No. 9,125,662, entitled “Multi-Axis Articulating and Rotating Surgical Tools,” issued Sep. 8, 2015, the disclosure of which is incorporated by reference herein, in its entirety; U.S. Pat. No. 8,820,605, entitled “Robotically-Controlled Surgical Instruments,” issued Sep. 2, 2014, the disclosure of which is incorporated by reference herein, in its entirety; U.S. Pub. No. 2019/0201077, entitled “Interruption of Energy Due to Inadvertent Capacitive Coupling,” published Jul. 4, 2019, the disclosure of which is incorporated by reference herein, in its entirety; U.S. Pub. No. 2012/0292367, entitled “Robotically-Controlled End Effector,” published on Nov. 11, 2012, now abandoned, the disclosure of which is incorporated by reference herein, in its entirety; and U.S. patent application Ser. No. 16/556,661, entitled “Ultrasonic Surgical Instrument with a Multi-Planar Articulating Shaft Assembly,” filed on Aug. 30, 2019, the disclosure of which is incorporated by reference herein, in its entirety.

Such instruments and robotic surgical systems may be further be incorporated into a surgical system for performing procedures in a surgical environment, such as surgical operating theaters or rooms in a healthcare facility. A sterile field is typically created around the patient and may include properly attired, scrubbed healthcare professions as well as desired furniture and/or fixtures. Examples of such surgical systems and associated features are disclosed in U.S. Pat. Pub. No. 2019/0201046, entitled “Method for Controlling Smart Energy Devices,” published on Jul. 4, 2019, the disclosure of which is incorporated by reference herein, in its entirety; U.S. Pat. Pub. No. 2019/0201080, entitled “Ultrasonic Energy Device Which Varies Pressure Applied by Clamp Arm to Provide Threshold Control Pressure at a Cut Progression Location,” published on Jul. 4, 2019, the disclosure of which is incorporated by reference herein, in its entirety; U.S. Pat. Pub. No. 2019/0201091, entitled “Radio Frequency Energy Device for Delivering Combined Electrical Signals,” published Jul. 4, 2019, the disclosure of which is incorporated by reference herein, in its entirety; U.S. Pat. Pub. No. 2019/0274717, entitled “Methods for Controlling Temperature in Ultrasonic Device,” published Sep. 12, 2019, the disclosure of which is incorporated by reference herein, in its entirety; and U.S. Pat. Pub. No. 2019/0207857, entitled “Surgical Network Determination of Prioritization of Communication, Interaction, or Processing Based on System or Device Needs,” published Jul. 4, 2019, the disclosure of which is incorporated by reference herein, in its entirety.

While several surgical instruments and systems have been made and used, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a block diagram of an example a computer-implemented interactive surgical system;

FIG. 2 depicts a top schematic view of an example of a surgical system for performing a surgical procedure in an operating room of a healthcare facility;

FIG. 3 depicts a side schematic view of an example of a surgical hub of the surgical system of FIG. 2;

FIG. 4 depicts a perspective view of a combination generator module with bipolar, ultrasonic, and monopolar contacts of the surgical system of FIG. 2;

FIG. 5 depicts a side schematic view of an exemplary generator and various examples of surgical instruments for use with the surgical system of FIG. 2;

FIG. 6 depicts a schematic view of an exemplary surgical visualization system including an imaging device and a surgical device;

FIG. 7 depicts a schematic diagram of an exemplary control system that may be used with the surgical visualization system of FIG. 6;

FIG. 8 depicts a flow chart of an exemplary method for determining the recovery capacity of at least one feature of a surgical instrument;

FIG. 9 depicts a flow chart of an exemplary assessment method for determining the recovery capacity of at least one feature of a surgical instrument;

FIG. 10 depicts a flow chart of an exemplary assessment method for determining the recovery capacity of at least one feature of a surgical instrument;

FIG. 11 depicts a flow chart of an exemplary assessment method for determining the recovery capacity of at least one feature of a surgical instrument;

FIG. 12 depicts a flow chart of an exemplary assessment method for determining the recovery capacity of at least one feature of a surgical instrument;

FIG. 13 depicts a perspective view of an assessment and cleaning port that may be utilized to assess the recovery capacity of at least one feature of surgical instrument and then clean the at least one feature of the surgical instrument;

FIG. 14A depicts a sectional view of the assessment and cleaning port of FIG. 13 with an end effector partially inserted;

FIG. 14B depicts a sectional view of the assessment and cleaning port of FIG. 13 with the end effector further inserted;

FIG. 15 depicts a perspective view of an end effector sheath;

FIG. 16A depicts a sectional view of the end effector sheath of FIG. 15 with an end effector adjacent to an entry of the sheath;

FIG. 16B depicts a sectional view of the end effector sheath of FIG. 15 with an end effector inserted into the sheath;

FIG. 17 depicts an top plan view of a surgical kit packaging; and

FIG. 18 depicts a perspective view of a removable cleaning kit of the surgical kit packaging of FIG. 17.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

For clarity of disclosure, the terms “proximal” and “distal” are defined herein relative to a human or robotic operator of the surgical instrument. The term “proximal” refers the position of an element closer to the human or robotic operator of the surgical instrument and further away from the surgical end effector of the surgical instrument. The term “distal” refers to the position of an element closer to the surgical end effector of the surgical instrument and further away from the human or robotic operator of the surgical instrument. In addition, the terms “upper,” “lower,” “top,” “bottom,” “above,” and “below,” are used with respect to the examples and associated figures and are not intended to unnecessarily limit the invention described herein.

### I. Example of a Surgical System

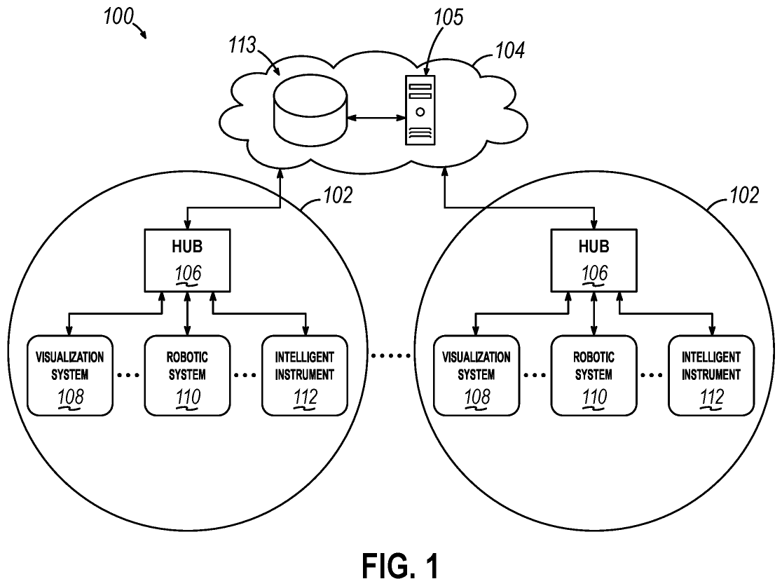

With respect to FIG. 1, a computer-implemented interactive surgical system (100) includes one or more surgical systems (102) and a cloud-based system (e.g., a cloud (104) that may include a remote server (113) coupled to a storage device (105)). Each surgical system (102) of the present example includes at least one surgical hub (106) in communication with cloud (104) that may include a remote server (113). In one example, as illustrated in FIG. 1, surgical system (102) includes a visualization system (108), a robotic system (110), and a handheld intelligent surgical instrument (112), which are configured to communicate with one another and/or hub (106). In some aspects, a surgical system (102) may include an M number of hubs (106), an N number of visualization systems (108), an O number of robotic systems (110), and a P number of handheld intelligent surgical instruments (112), where M, N, O, and P are integers greater than or equal to one. In any case, any suitable combination of features provided below may be incorporated into an exemplary surgical system, such as surgical system (100), and used in the surgical theater in order to perform a desired surgical procedure as would be apparent to one skilled in the art in view of the teachings herein.

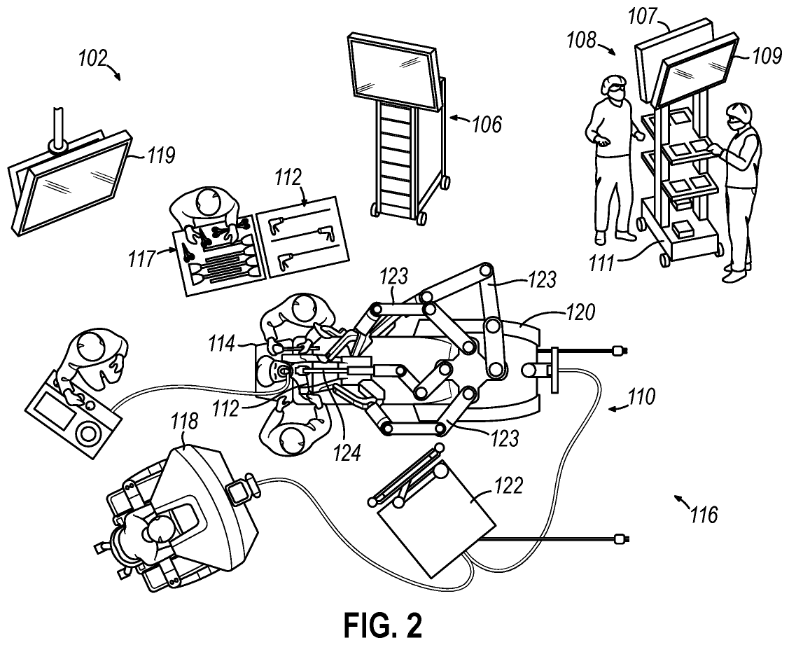

FIG. 2 depicts an example of a surgical system (102) being used to perform a surgical procedure on a patient who is lying down on an operating table (114) in a surgical operating room (116). A robotic system (110) is used in the surgical procedure as a part of surgical system (102). Robotic system (110) includes a surgeon's console (118), a patient side cart (120) (surgical robot), and a surgical robotic hub (122). Patient side cart (120) can manipulate at least one removably coupled surgical tool (117) with any one of a plurality of surgical arms (123) through a minimally invasive incision in the body of the patient while the surgeon views the surgical site through console (118). An image of the surgical site can be obtained by a medical imaging device (124), which can be manipulated by patient side cart (120) to orient imaging device (124). Robotic hub (122) can be used to process the images of the surgical site for subsequent display to the surgeon through console (118).

Other types of robotic systems can be readily adapted for use with surgical system (102). Various examples of robotic systems and surgical tools that are suitable for use with the present disclosure are described in U.S. Provisional Patent Application Ser. No. 62/611,339, entitled “Robot Assisted Surgical Platform,” filed Dec. 28, 2017, the disclosure of which is herein incorporated by reference in its entirety.

Various examples of cloud-based analytics that are performed by cloud (104, and are suitable for use with the present disclosure, are described in U.S. Provisional Patent Application Ser. No. 62/611,340, entitled Cloud-Based Medical Analytics,” filed Dec. 28, 2017, the disclosure of which is herein incorporated by reference in its entirety.

In various aspects, imaging device (124) includes at least one image sensor and one or more optical components. Suitable image sensors include, but are not limited to, Charge-Coupled Device (CCD) sensors and Complementary Metal-Oxide Semiconductor (CMOS) sensors. In various aspects, imaging device (124) is configured for use in a minimally invasive procedure. Examples of imaging devices suitable for use with the present disclosure include, but not limited to, an arthroscope, angioscope, bronchoscope, choledochoscope, colonoscope, cytoscope, duodenoscope, enteroscope, esophagogastro-duodenoscope (gastroscope), endoscope, laryngoscope, nasopharyngo-neproscope, sigmoidoscope, thoracoscope, and ureteroscope. Some aspects of spectral and multispectral imaging are described in greater detail under the heading “Advanced Imaging Acquisition Module” in U.S. Provisional Patent Application Ser. No. 62/611,341, entitled “Interactive Surgical Platform,” filed Dec. 28, 2017, the disclosure of which is herein incorporated by reference in its entirety.

Strict sterilization of the operating room and surgical equipment is required during any surgery. The strict hygiene and sterilization conditions required in a “surgical theater,” i.e., an operating or treatment room, necessitate the highest possible sterility of all medical devices and equipment. Part of that sterilization process is the need to sterilize anything that comes in contact with the patient or penetrates the sterile field. It will be appreciated that the sterile field may be considered a specified area, such as within a tray or on a sterile towel, that is considered free of microorganisms, or the sterile field may be considered an area, immediately around a patient, who has been prepared for a surgical procedure. The sterile field may include the scrubbed team members, who are properly attired, and all furniture and fixtures in the area.

In addition to the introduction of any features of surgical system (100), furniture, or fixtures into the sterile field requiring sterilization, additional complications may result from removal of these features from the sterile field, particularly when such features may have contacted, or presumed to have contacted, the patient, including any tissues and/or fluids associated with the surgical procedure. Such contamination of these features from the patient often requires special consideration during or after the surgical procedure, particularly when processing these features for disposal, reuse, or remanufacturing as desired. In one example, surgical system (100) and/or healthcare professionals associated with the surgical procedure may be specifically equipped to address such processing as discussed below in greater detail.

As illustrated in FIG. 2, a primary display (119) is positioned in the sterile field to be visible to an operator at operating table (114). In addition, a visualization tower (111) is positioned outside the sterile field. Visualization tower (111) includes a first non-sterile display (107) and a second non-sterile display (109), which face away from each other. Visualization system (108), guided by hub (106), is configured to utilize displays (107, 109, 119) to coordinate information flow to operators inside and outside the sterile field. For example, hub (106) may cause visualization system (108) to display a snapshot of a surgical site, as recorded by imaging device (124), on a non-sterile display (107) or (109), while maintaining a live feed of the surgical site on the primary display (119). The snapshot on non-sterile display (107) or display (109) can permit a non-sterile operator to perform a diagnostic step relevant to the surgical procedure, for example.

In one aspect, hub (106) is also configured to route a diagnostic input or feedback entered by a non-sterile operator at visualization tower (111) to primary display (119) within the sterile field, where it can be viewed by a sterile operator at the operating table. In one example, the input can be in the form of a modification to the snapshot displayed on non-sterile display (107) or display (109), which can be routed to primary display (119) by hub (106).

Referring to FIG. 2, a surgical instrument (112) is being used in the surgical procedure as part of surgical system (102). Hub (106) is also configured to coordinate information flow to a display of the surgical instrument (112) such as in, for example, U.S. Provisional Patent Application Ser. No. 62/611,341, entitled “Interactive Surgical Platform,” filed Dec. 28, 2017, the disclosure of which is herein incorporated by reference in its entirety. A diagnostic input or feedback entered by a non-sterile operator at visualization tower (111) can be routed by hub (106) to surgical instrument display (115) within the sterile field, where it can be viewed by the operator of surgical instrument (112). Example surgical instruments that are suitable for use with surgical system (102) are described under the heading “Surgical Instrument Hardware” and in U.S. Provisional Patent Application Ser. No. 62/611,341, entitled “Interactive Surgical Platform,” filed Dec. 28, 2017, the disclosure of which is herein incorporated by reference in its entirety, for example.

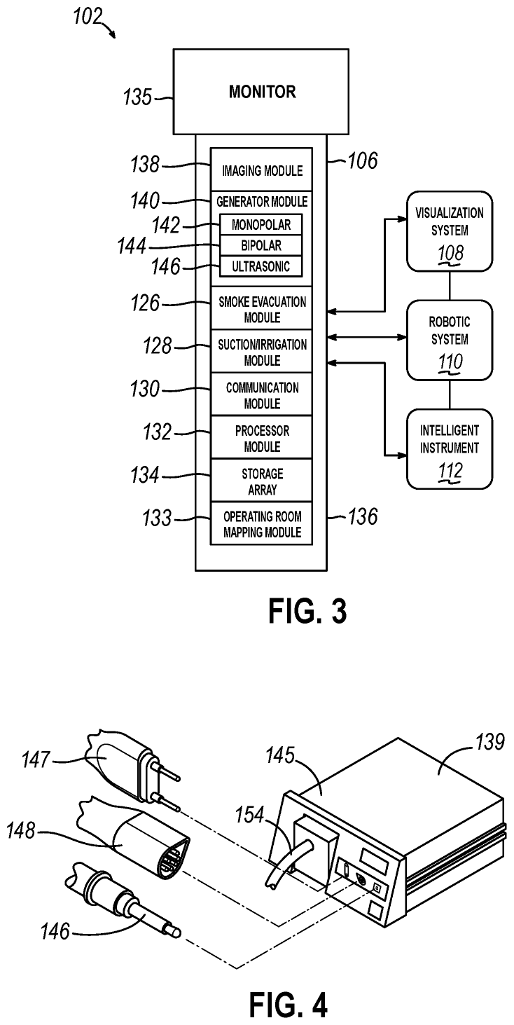

Referring now to FIG. 3, a hub (106) is depicted in communication with a visualization system (108), a robotic system (110), and a handheld intelligent surgical instrument (112). Hub (106) includes a hub display (135), an imaging module (138), a generator module (140), a communication module (130), a processor module (132), and a storage array (134). In certain aspects, as illustrated in FIG. 3, hub (106) further includes a smoke evacuation module (126), a suction/irrigation module (128), and/or an operating room mapping module (133).

During a surgical procedure, energy application to tissue, for sealing and/or cutting, is generally associated with smoke evacuation, suction of excess fluid, and/or irrigation of the tissue. Fluid, power, and/or data lines from different sources are often entangled during the surgical procedure. Valuable time can be lost addressing this issue during a surgical procedure. Detangling the lines may necessitate disconnecting the lines from their respective modules, which may require resetting the modules. The hub modular enclosure (136) offers a unified environment for managing the power, data, and fluid lines, which reduces the frequency of entanglement between such lines.

Referring to FIGS. 3-4, aspects of the present disclosure are presented for a hub modular enclosure (136) that allows the modular integration of a generator module (140), a smoke evacuation module (126), and a suction/irrigation module (128). Hub modular enclosure (136) further facilitates interactive communication between modules (140, 126, 128). As shown in FIG. 4, generator module (140) can be a generator module with integrated monopolar, bipolar, and ultrasonic components supported in a single housing unit (139) slidably insertable into hub modular enclosure (136). As illustrated in FIG. 4, generator module (140) can be configured to connect to a monopolar device (146), a bipolar device (147), and an ultrasonic device (148). Alternatively, generator module (140) may comprise a series of monopolar, bipolar, and/or ultrasonic generator modules that interact through hub modular enclosure (136). Hub modular enclosure (136) can be configured to facilitate the insertion of multiple generators and interactive communication between the generators docked into the hub modular enclosure (136) so that the generators would act as a single generator.

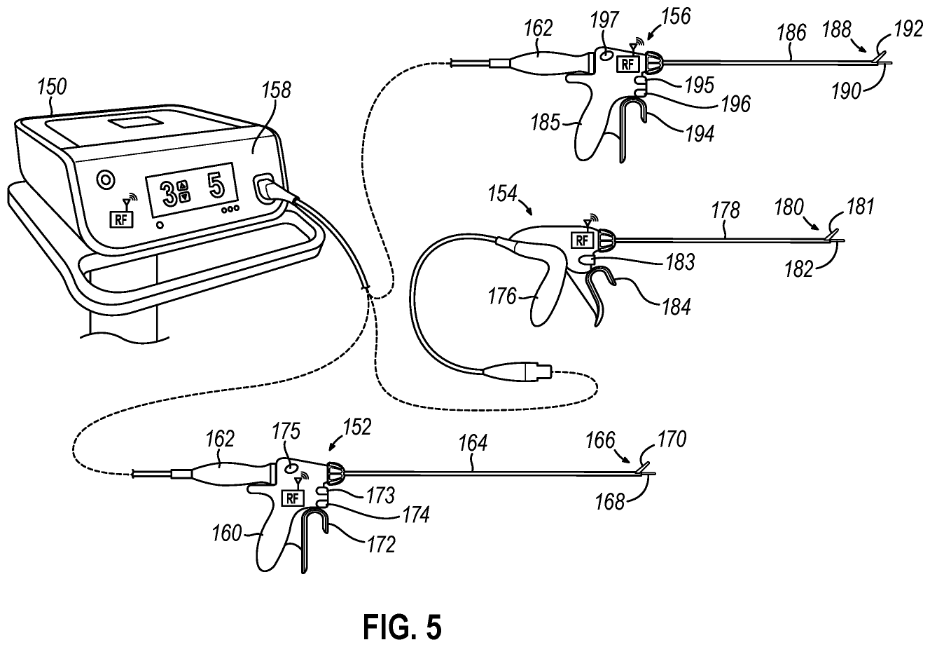

FIG. 5 illustrates one form of a generator (150) and various surgical instruments (152, 154, 156) usable therewith, where surgical instrument (152) is an ultrasonic surgical instrument (152), surgical instrument (154) is an RF electrosurgical instrument (154), and multifunction surgical instrument (156) is a combination ultrasonic/RF electrosurgical instrument (156). Generator (150) is configurable for use with a variety of surgical instruments. According to various forms, generator (150) may be configurable for use with different surgical instruments of different types including, for example, ultrasonic surgical instruments (152), RF electrosurgical instruments (154), and multifunction surgical instruments (156) that integrate RF and ultrasonic energies delivered simultaneously from generator (150). Although generator (150) of the present example in FIG. 5 is shown separate from surgical instruments (152, 154, 156), generator (150) may alternatively be formed integrally with any of surgical instruments (152, 154, 156) to form a unitary surgical system. Generator (150) comprises an input device (158) located on a front panel of generator (150) console. Input device (158) may comprise any suitable device that generates signals suitable for programming the operation of generator (150). Generator (150) may be configured for wired or wireless communication.

Generator (150) of the present example is configured to drive multiple surgical instruments (152, 154, 156). One example of such surgical instrument is ultrasonic surgical instrument (152) and comprises a handpiece (160), an ultrasonic transducer 162, a shaft assembly (164), and an end effector (166). End effector (166) includes an ultrasonic blade (168) acoustically coupled to ultrasonic transducer (162) and a clamp arm (170). Handpiece (160) has a trigger (172) to operate clamp arm (170) and a combination of toggle buttons (173, 174, 175) to energize and drive ultrasonic blade (168) or other function. Toggle buttons (173, 174, 175) can be configured to energize ultrasonic transducer (162) with generator (150).

Generator (150) also is configured to drive another example of surgical instrument (154). RF electrosurgical instrument (154) includes a handpiece (176), a shaft assembly (178), and an end effector (180). End effector (180) includes electrodes in clamp arms (181, 182) and return through an electrical conductor portion of shaft assembly (178). Electrodes are coupled to and energized by a bipolar energy source within generator (150). Handpiece (176) includes a trigger (183) to operate clamp arms (181, 182) and an energy button (184) to actuate an energy switch to energize electrodes in end effector (180).

Generator (150) also is configured to drive multifunction surgical instrument (156). Multifunction surgical instrument (156) includes a handpiece (185), a shaft assembly (186), and an end effector (188). End effector (188) has an ultrasonic blade (190) and a clamp arm (192). Ultrasonic blade (190) is acoustically coupled to ultrasonic transducer (162). Handpiece (185) has a trigger (194) to operate clamp arm (192) and a combination of toggle buttons (195, 196, 197) to energize and drive ultrasonic blade (190) or other function. Toggle buttons (195, 196, 197) can be configured to energize ultrasonic transducer (162) with generator (150) and energize ultrasonic blade (190) with a bipolar energy source also contained within generator (150). It will be appreciated that handpieces (160, 176, 185) may be replaced with a robotically controlled instrument for incorporating one or more aspects of surgical instruments (152, 154, 156). Accordingly, the term “handpiece” should not be limited to this context and to handheld use.

As used throughout this description, the term “wireless” and its derivatives may be used to describe circuits, devices, systems, methods, techniques, communications channels, etc., that may communicate data through the use of modulated electromagnetic radiation through a non-solid medium. The term does not imply that the associated devices do not contain any wires, although in some aspects they might not. The communication module may implement any of a number of wireless or wired communication standards or protocols, including but not limited to Wi-Fi (IEEE 802.11 family), WMAX (IEEE 802.16 family), IEEE 802.20, long term evolution (LTE), Ev-DO, HSPA+, HSDPA+, HSUPA+, EDGE, GSM, GPRS, CDMA, TDMA, DECT, Bluetooth, Ethernet derivatives thereof, as well as any other wireless and wired protocols that are designated as 3G, 4G, 5G, and beyond. The computing module may include a plurality of communication modules. For instance, a first communication module may be dedicated to shorter range wireless communications such as Wi-Fi and Bluetooth and a second communication module may be dedicated to longer range wireless communications such as GPS, EDGE, GPRS, CDMA, WiMAX, LTE, Ev-DO, and others.

As used herein a processor or processing unit is an electronic circuit which performs operations on some external data source, usually memory or some other data stream. The term is used herein to refer to the central processor (central processing unit) in a system or computer systems (especially systems on a chip (SoCs)) that combine a number of specialized “processors.”

As used herein, a system on a chip or system on chip (SoC or SOC) is an integrated circuit (also known as an “IC” or “chip”) that integrates all components of a computer or other electronic systems. It may contain digital, analog, mixed-signal, and often radiofrequency functions, all on a single substrate. A SoC integrates a microcontroller (or microprocessor) with advanced peripherals like graphics processing unit (GPU), Wi-Fi module, or coprocessor. A SoC may or may not contain built-in memory.

As used herein, a microcontroller or controller is a system that integrates a microprocessor with peripheral circuits and memory. A microcontroller (or MCU for microcontroller unit) may be implemented as a small computer on a single integrated circuit. It may be similar to a SoC; an SoC may include a microcontroller as one of its components. A microcontroller may contain one or more core processing units (CPUs) along with memory and programmable input/output peripherals. Program memory in the form of Ferroelectric RAM, NOR flash or OTP ROM is also often included on chip, as well as a small amount of RAM. Microcontrollers may be employed for embedded applications, in contrast to the microprocessors used in personal computers or other general purpose applications consisting of various discrete chips.

As used herein, the term controller or microcontroller may be a stand-alone IC or chip device that interfaces with a peripheral device. This may be a link between two parts of a computer or a controller on an external device that manages the operation of (and connection with) that device. Modular devices include the modules (as described in connection with FIG. 3, for example) that are receivable within a surgical hub and the surgical devices or instruments that can be connected to the various modules in order to connect or pair with the corresponding surgical hub. The modular devices include, for example, intelligent surgical instruments, medical imaging devices, suction/irrigation devices, smoke evacuators, energy generators, ventilators, insufflators, and displays. The modular devices described herein can be controlled by control algorithms. The control algorithms can be executed on the modular device itself, on the surgical hub to which the particular modular device is paired, or on both the modular device and the surgical hub (e.g., via a distributed computing architecture). In some exemplifications, the modular devices' control algorithms control the devices based on data sensed by the modular device itself (i.e., by sensors in, on, or connected to the modular device). This data can be related to the patient being operated on (e.g., tissue properties or insufflation pressure) or the modular device itself (e.g., the rate at which a knife is being advanced, motor current, or energy levels). For example, a control algorithm for a surgical stapling and cutting instrument can control the rate at which the instrument's motor drives its knife through tissue according to resistance encountered by the knife as it advances.

### II. Exemplary Surgical Visualization System

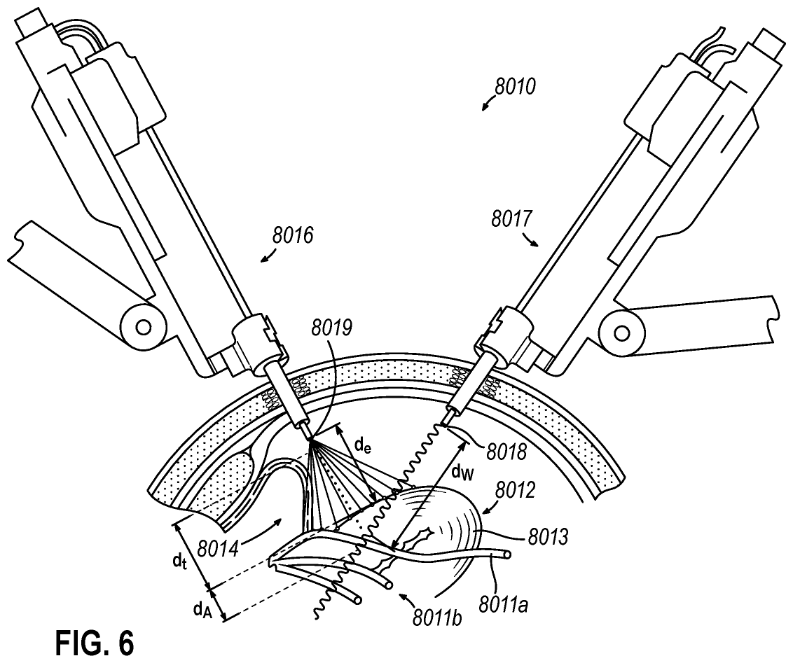

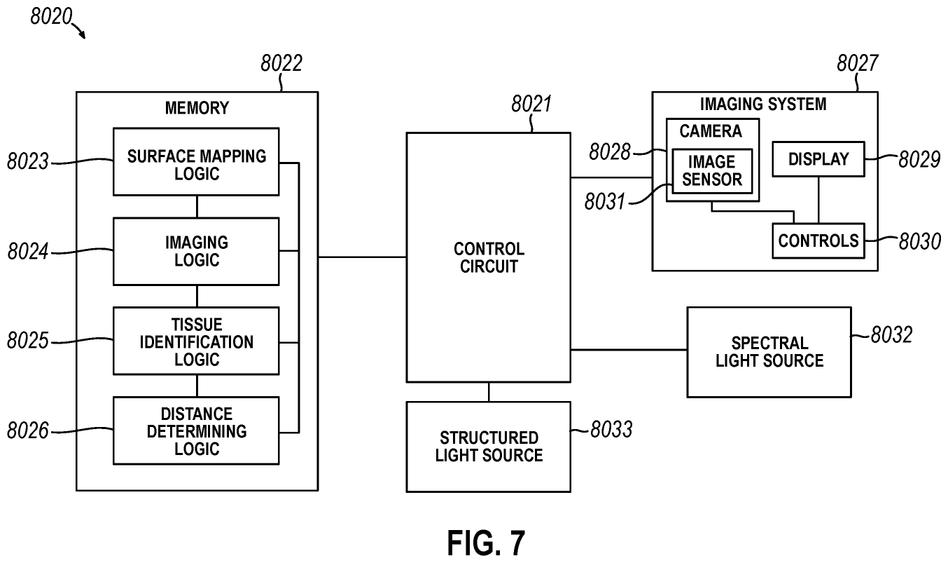

FIGS. 6-7 depicts a schematic view of a surgical visualization system (8010) and a schematic diagram of a control system (8020) that may be used in conjunction with each other, according to at least one aspect of the present disclosure. Surgical visualization system (8010) and control system (8020) may be readily incorporated into computer-implemented interactive surgical system (100) described above. For example, surgical visualization system (8010) may be used in replacement of imaging device (124) described above; while control system (8020) may be used in replacement of imaging module (138) described above. Surgical visualization system (8010) may create a visual representation of a critical structure (8011a, 8011b) within an anatomical field.

Critical structures (8011a, 8011b) may be any anatomical structures of interest or any foreign structure in the anatomical field. In one aspect, a critical structure (8011a, 8011b) may be embedded in tissue. Stated differently, a critical structure (8011a, 8011b) may be positioned below a surface of the tissue. In such instances, the tissue conceals the critical structure (8011a, 8011b) from the clinician's view. A critical structure (8011a, 8011b) may also be obscured from the view of an imaging device by the tissue. The tissue may be fat, connective tissue, adhesions, and/or organs, for example. In other instances, a critical structure (8011a, 8011b) may be partially obscured from view. Surgical visualization system (8010) is shown being utilized intraoperatively to identify and facilitate avoidance of certain critical structures, such as a ureter (8011a) and vessels (8011b) in an organ (8012) (the uterus in this example), that are not visible on a surface (8013) of the organ (8012).

With continuing reference to FIG. 6, surgical visualization system (8010) incorporates tissue identification and geometric surface mapping in combination with a distance sensor system (8014). In combination, these features of surgical visualization system (8010) may determine a position of critical structure (8011a, 8011b) within the anatomical field and/or the proximity of a surgical device (8016) to surface (8013) of the visible tissue and/or to critical structure (8011a, 8011b). Surgical device (8016) may be substantially similar to surgical instrument/tool (112, 117, 152, 154, 156) described herein. As also described herein, surgical visualization system (8010) may be configured to achieve identification of one or more critical structures (8011a, 8011b) and/or the proximity of surgical device (8016) to critical structure(s) (8011a, 8011b).

The depicted surgical visualization system (8010) includes an imaging system that includes an imaging device (8017), such as a camera of a scope, for example, that is configured to provide real-time views of the surgical site. In various instances, imaging device (8017) includes a spectral camera (e.g., a hyperspectral camera, multispectral camera, a fluorescence detecting camera, or selective spectral camera), which is configured to detect reflected or emitted spectral waveforms and generate a spectral cube of images based on the molecular response to the different wavelengths. Views from imaging device (8017) may be provided to a clinician; and, in various aspects of the present disclosure, may be augmented with additional information based on the tissue identification, landscape mapping, and input from a distance sensor system (8014). In such instances, a surgical visualization system (8010) includes a plurality of subsystems—an imaging subsystem, a surface mapping subsystem, a tissue identification subsystem, and/or a distance determining subsystem. These subsystems may cooperate to intraoperatively provide advanced data synthesis and integrated information to the clinician(s).

Imaging device (8017) of the present example includes an emitter (8018), which is configured to emit spectral light in a plurality of wavelengths to obtain a spectral image of hidden structures, for example. Imaging device (8017) may also include a three-dimensional camera and associated electronic processing circuits in various instances. In one aspect, emitter (8018) is an optical waveform emitter that is configured to emit electromagnetic radiation (e.g., near-infrared radiation (NIR) photons) that may penetrate surface (8013) of tissue (8012) and reach critical structure(s) (8011a, 8011b). Imaging device (8017) and optical waveform emitter (8018) thereon may be positionable by surgical arms (123) (see FIG. 2) or a surgeon manually operating imaging device (8017). A corresponding waveform sensor (e.g., an image sensor, spectrometer, or vibrational sensor, etc.) on imaging device (8017) may be configured to detect the effect of the electromagnetic radiation received by the waveform sensor.

The wavelengths of the electromagnetic radiation emitted by optical waveform emitter (8018) may be configured to enable the identification of the type of anatomical and/or physical structure, such as critical structure(s) (8011a, 8011b). The identification of critical structure(s) (8011a, 8011b) may be accomplished through spectral analysis, photo-acoustics, fluorescence detection, and/or ultrasound, for example. In one aspect, the wavelengths of the electromagnetic radiation may be variable. The waveform sensor and optical waveform emitter (8018) may be inclusive of a multispectral imaging system and/or a selective spectral imaging system, for example. In other instances, the waveform sensor and optical waveform emitter (8018) may be inclusive of a photoacoustic imaging system, for example. In other instances, optical waveform emitter (8018) may be positioned on a separate surgical device from imaging device (8017).

The depicted surgical visualization system (8010) also includes an emitter (8019), which is configured to emit a pattern of light, such as stripes, grid lines, and/or dots, to enable the determination of the topography or landscape of surface (8013). For example, projected light arrays may be used for three-dimensional scanning and registration on surface (8013). The projected light arrays may be emitted from emitter (8019) located on surgical device (8016) and/or imaging device (8017), for example. In one aspect, the projected light array is employed to determine the shape defined by surface (8013) of the tissue (8012) and/or the motion of surface (8013) intraoperatively. Imaging device (8017) is configured to detect the projected light arrays reflected from surface (8013) to determine the topography of surface (8013) and various distances with respect to surface (8013).

The depicted surgical visualization system (8010) also includes distance sensor system (8014) configured to determine one or more distances at the surgical site. In one aspect, distance sensor system (8014) may include a time-of-flight distance sensor system that includes an emitter, such as structured light emitter (8019); and a receiver (not shown), which may be positioned on surgical device (8016). In other instances, the time-of-flight emitter may be separate from structured light emitter (8019). In one general aspect, the emitter portion of time-of-flight distance sensor system (8014) may include a laser source and the receiver portion of time-of-flight distance sensor system (8014) may include a matching sensor. Time-of-flight distance sensor system (8014) may detect the “time of flight,” or how long the laser light emitted by structured light emitter (8019) has taken to bounce back to the sensor portion of the receiver. Use of a very narrow light source in structured light emitter (8019) may enable distance sensor system (8014) to determine the distance to surface (8013) of the tissue (8012) directly in front of distance sensor system (8014).

Referring still to FIG. 6, distance sensor system (8014) may be employed to determine an emitter-to-tissue distance (dₑ) from structured light emitter (8019) to surface (8013) of the tissue (12). A device-to-tissue distance (dₜ) from the distal end of surgical device (8016) to surface (8013) of the tissue (12) may be obtainable from the known position of emitter (8019) on the shaft of surgical device (8016) relative to the distal end of surgical device (8016). In other words, when the distance between emitter (8019) and the distal end of surgical device (8016) is known, the device-to-tissue distance (dₜ) may be determined from the emitter-to-tissue distance (dₑ). In certain instances, the shaft of surgical device (8016) may include one or more articulation joints; and may be articulatable with respect to emitter (8019) and the jaws. The articulation configuration may include a multi-joint vertebrae-like structure, for example. In certain instances, a three-dimensional camera may be utilized to triangulate one or more distances to surface (8013).

As described above, surgical visualization system (8010) may be configured to determine the emitter-to-tissue distance (dₑ) from emitter (8019) on surgical device (8016) to surface (8013) of a uterus (12) via structured light. Surgical visualization system (8010) is configured to extrapolate a device-to-tissue distance (dₜ) from surgical device (8016) to the surface (13) of the uterus (12) based on emitter-to-tissue distance (dₑ). Surgical visualization system (10) is also configured to determine a tissue-to-ureter distance (d_{A}) from a ureter (11a) to the surface (13) and a camera-to-ureter distance (d_{w}), from imaging device (17) to the ureter (11a). Surgical visualization system (10) may determine the camera-to-ureter distance (d_{w}), with spectral imaging and time-of-flight sensors, for example. In various instances, a surgical visualization system (10) may determine (e.g., triangulate) a tissue-to-ureter distance (d_{A}) (or depth) based on other distances and/or the surface mapping logic described herein.

FIG. 7 is a schematic diagram of a control system (8020), which may be utilized with a surgical visualization system (8010). The depicted control system (8020) includes a control circuit (8021) in signal communication with a memory (8022). Memory (8022) stores instructions executable by control circuit (8021) to determine and/or recognize critical structures (e.g., critical structures (8011a, 8011b) depicted in FIG. 6), determine and/or compute one or more distances and/or three-dimensional digital representations, and to communicate certain information to one or more clinicians. For example, memory (8022) stores surface mapping logic (8023), imaging logic (8024), tissue identification logic (8025), or distance determining logic (8026) and/or any combinations of logic (8023, 8024, 8025, 8026). Control system (8020) also includes an imaging system (8027) having one or more cameras (8028) (like imaging device (8017) depicted in FIG. 6), one or more displays (8029), one or more controls (8030) or any combinations of these elements. The one or more cameras (8028) may include one or more image sensors (8031) to receive signals from various light sources emitting light at various visible and invisible spectra (e.g., visible light, spectral imagers, three-dimensional lens, among others). Display (8029) may include one or more screens or monitors for depicting real, virtual, and/or virtually-augmented images and/or information to one or more clinicians.

In various aspects, a main component of camera (8028) includes an image sensor (8031). Image sensor (8031) may include a Charge-Coupled Device (CCD) sensor, a Complementary Metal Oxide Semiconductor (CMOS) sensor, a short-wave infrared (SWIR) sensor, a hybrid CCD/CMOS architecture (sCMOS) sensor, and/or any other suitable kind(s) of technology. Image sensor (8031) may also include any suitable number of chips.

Control system (8020) also includes a spectral light source (8032) and a structured light source (8033). In certain instances, a single source may be pulsed to emit wavelengths of light in spectral light source (8032) range and wavelengths of light in structured light source (8033) range. Alternatively, a single light source may be pulsed to provide light in the invisible spectrum (e.g., infrared spectral light) and wavelengths of light on the visible spectrum. Spectral light source (8032) may include a hyperspectral light source, a multi spectral light source, a fluorescence excitation light source, and/or a selective spectral light source, for example. In various instances, tissue identification logic (8025) may identify critical structure(s) via data from a spectral light source (8032) received by the image sensor (8031) portion of camera (8028). Surface mapping logic (8023) may determine the surface contours of the visible tissue based on reflected structured light. With time-of-flight measurements, distance determining logic (8026) may determine one or more distance(s) to the visible tissue and/or critical structure(s) (8011a, 8011b). One or more outputs from surface mapping logic (8023), tissue identification logic (8025), and/or distance determining logic (8026) may be provided to imaging logic (8024) and combined, blended, and/or overlaid to be conveyed to a clinician via display (8029) of imaging system (8027).

Surgical visualization system (8010) and control system (8020) may include the teachings of U.S. patent application Ser. No. 17/373,593, entitled “Endoscope with Synthetic Aperture Multispectral Camera Array,” filed on Aug. 14, 2021, the disclosure of which is incorporated by reference herein.

### III. Exemplary Method for Instrument Assessment of Recovery Capacity

As mentioned above, in some instances after exemplary use, features of surgical instrument/tool (112, 117, 152, 154, 156, 8016) may be processed for disposal, reuse, and/or remanufacturing. Suitable reuse and/or remanufacturing of features of surgical instrument/tool (112, 117, 152, 154, 156, 8016) may be referred to as recovering features of instrument/tool (112, 117, 152, 154, 156, 8016). In some instances, recovered features of surgical instrument/tool (112, 117, 152, 154, 156, 8016) may be reused by being incorporated into rebuilding a reprocessed product.

Processing features of a used surgical instrument/tool (112, 117, 152, 154, 156, 8016) for suitable recovery (i.e., reuse and/or remanufacturing) in accordance with the description herein may require significant resources (e.g., effort, time, and money). Additionally, in some instances, features of a used surgical instrument/tool (112, 117, 152, 154, 156, 8016) that are originally intended for suitable recovery may experience an undesirable amount of damage and/or performance degradation during exemplary use such that recovering a specific feature of surgical instrument/tool (112, 117, 152, 154, 156, 8016) is no longer feasible. When features originally intended for suitable recovery experience an undesirable amount of damage and/or performance degradation, significant resources may be utilized in an unsuccessful attempt to recover such features. Therefore, it may be desirable to determine if at least some features of a specific used surgical instrument/tool (112, 117, 152, 154, 156, 8016) have the capacity of being suitably recovered after exemplary use, but prior to investing significant resources.

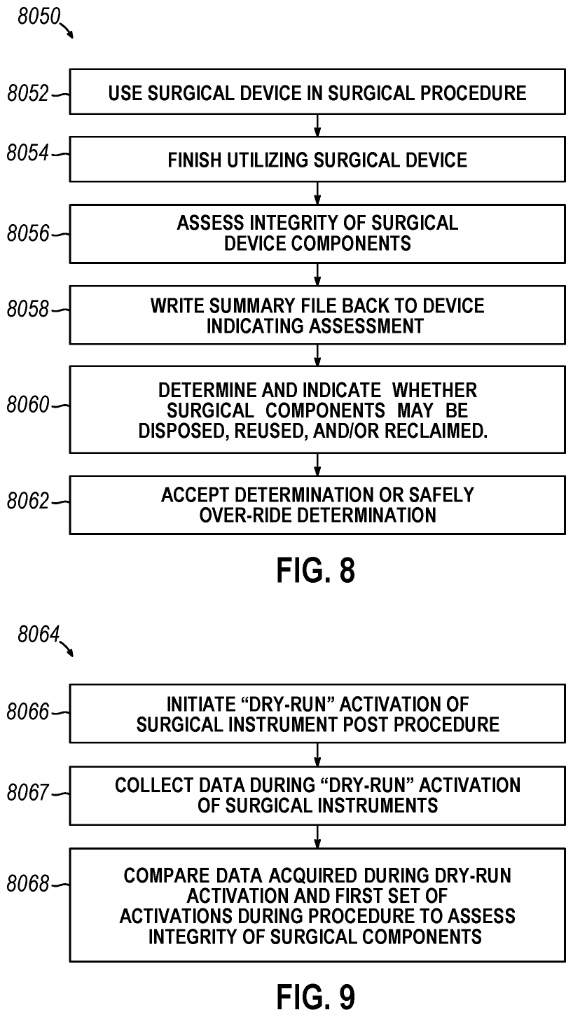

FIG. 8 shows an exemplary method (8050) of determining recovery capacity for at least some features of a surgical instrument/tool (112, 117, 152, 154, 156, 8016). In some non-limiting aspects of the disclosure, features of surgical instrument/tool (112, 117, 152, 154, 156, 8016) that method (8050) may be used to determine the recovery capacity of include, but are not limited to: suitable electrical components, handpiece (160, 176, 185), shaft assembly (164, 178, 186), end effector (166, 180, 188), ultrasonic blade (168, 190), clamp arm (170, 181, 182, 192), trigger (183, 194), toggle button (173, 174, 175, 195, 196, 197), suitable portions of the above mentioned components, or any other suitable components as would be apparent to one skilled in the art in view of the teachings herein.

First, an operator may use (8052) surgical instrument/tool (112, 117, 152, 154, 156, 8016) in order to perform a suitable medical procedure on a patient in accordance with the description herein. As mentioned above, during use (8052), surgical instrument/tool (112, 117, 152, 154, 156, 8016) may be suitably coupled to, and in communication with, hub (106), generator module (140), patient side cart (120), surgical robot hub (122), surgical visualization system (8010), control system (8020), etc. Therefore, suitable devices of computer-implemented interactive surgical system (100) may be able to measure, collect, and/or store various data related to the operation of surgical instrument/tool (112, 117, 152, 154, 156, 8016) in accordance with the description herein.

Next, the operator may finish (8054) utilizing surgical instrument/tool (112, 117, 152, 154, 156, 8016). The operator may interact with suitable components of interactive surgical system (100) in order to indicate that surgical instrument/tool (112, 117, 152, 154, 156, 8016) is finished being utilized for a specific surgical procedure. In some non-limiting aspects of the disclosure, the operator (or any other suitable individual) may press a button of surgical system (100) in order to indicate that that surgical instrument/tool (112, 117, 152, 154, 156, 8016) is finished being used and/or the medical procedure has been completed. For example, such a button may be located on generator module (140). Of course, any other suitable means may be utilized in order to indicate that surgical instrument/tool (112, 117, 152, 154, 156, 8016) is finished being used as would be apparent to one skilled in the art in view of the teachings herein.

As mentioned above, suitable components of surgical system (100) may measure, collect, and/or store various data related to the operation of surgical instrument/tool (112, 117, 152, 154, 156, 8016). Once surgical instrument/tool (112, 117, 152, 154, 156, 8016) is finished (8054) being utilized, generator module (140), hub (106), control system (8020), or any other suitable component(s) may be utilized in order to assess (8056) the integrity and/or recovery capacity of at least one feature of surgical instrument/tool (112, 117, 152, 154, 156, 8016). Any suitable components and/or methods may be utilized to assess (8056) the integrity and/or recovery capacity of at least one feature of surgical instrument/tool (112, 117, 152, 154, 156, 8016) as would be apparent to one skilled in the art in view of the teachings herein.

In some aspects of the disclosure, after assessing (8056) the integrity and/or recovery capacity of at least one feature of surgical instrument/tool (112, 117, 152, 154, 156, 8016), generator module (140), hub (106), control system (8020), or any other suitable component(s) may write (8058) a summary file of the assessment (8056) back to suitable electrical components of surgical instrument/tool (112, 117, 152, 154, 156, 8016). Additionally, or alternatively, generator module (140), hub (106), control system (8020), or any other suitable component(s) may also determine and indicate (8060) whether specific features of surgical instrument/tool (112, 117, 152, 154, 156, 8016) have the capacity of being recovered or if such features should be discarded/disposed. Therefore, in some instances, a user may directly view on generator module (140), hub (106), control system (8020), or any other suitable component(s) whether certain features of surgical instrument/tool (112, 117, 152, 154, 156, 8016) have the capacity to be recovered. In some instances, generator module (140), hub (106), control system (8020), etc., may then be used to then retrieve and display proper disposal/recovery instructions.

In instances where a summary file is written (8058) back to surgical instrument/tool (112, 117, 152, 154, 156, 8016), such a summary file may include a synopsis of the assessment (8056) and a recommendation on whether features of surgical instrument/tool (112, 117, 152, 154, 156, 8016) should be disposed or recovered. The summary file written (8058) back to surgical instrument/tool (112, 117, 152, 154, 156, 8016) may be utilized during the disassembly process as would be apparent to one skilled in the art in view of the teachings herein. For example, in instances where an electronic disassembly assistance device is utilized, such as a smart phone or a tablet, such a device may communicate with surgical instrument/tool (112, 117, 152, 154, 156, 8016) in order to retrieve the summary file. The electronic disassembly assistance device may then utilize the summary file to retrieve or generate instructions related to proper recovery and/or disposal. In some instances, the electronic disassembly assistance device may communicate with generator module (140), hub (106), control system (8020), etc., in order to retrieve or generate such instructions.

In some instances, a user may be provided with the option to accept or safely over-ride (8062) the determination (8060) made as to whether surgical instrument/tool (112, 117, 152, 154, 156, 8016) should be disposed or recovered. In instances where a user decides to over-ride a determination (8060) that a feature of surgical instrument/tool (112, 117, 152, 154, 156, 8016) should be disposed, electronic disassembly assistance device or any other feature configured to display instructions may then display recovery instructions rather than disposal instructions. Additionally, electronic disassembly assistance device or any other suitable display feature may also display the implication and/or potential consequences over-riding (8062) the system's determination (8060).

However, in some instance where the assessed (8056) damage to features of surgical instrument/tool (112, 117, 152, 154, 156, 8016) exceeds a predetermined, ultimate limit, the disposal determination (8060) of such a feature may become an unassailable determination that is not over-ridable. For example, in instances where the assessed (8060) feature is ultrasonic blade (168), if assessment (8060) determines blade damage is coming to a threshold that, if exceeded, the instrument will fail in an unsafe manner; the system could prevent the user from over-riding (8062) the determination (8060) and also prevent further use of the specific ultrasound blade (168). In some instances, the system could prevent over-riding (8062) in a limiting manner rather than an absolute manner such that portions of features are prevented from operating or features are prevented from being used at a predetermined, maximum power level.

Assessment (8056) may be performed at any suitable time after surgical instrument/tool (112, 117, 152, 154, 156, 8016) is finished being utilized as would be apparent to one skilled in the art in view of the teachings herein. Therefore, method (8050) may be utilized within the surgical theater in order to assess if specific features of surgical instrument/tool (112, 117, 152, 154, 156, 8016) should be discarded immediately or if such features show any potential for being recovered.

As mentioned above, any suitable components and/or methods may be utilized to assess (8056) the integrity and/or recovery capacity of at least one feature of surgical instrument/tool (112, 117, 152, 154, 156, 8016) as would be apparent to one skilled in the art in view of the teachings herein. FIG. 9 shows one exemplary assessment method (8064) that may be used to assess the recovery capacity of suitable features of surgical instrument/tool (112, 117, 152, 154, 156, 8016). Therefore, it should be understood that assessment method (8064) may be readily incorporated into method (8050) described above.

First, a user may initiate a “dry-run” activation (8066) of surgical instrument/tool (112, 117, 152, 154, 156, 8016) after surgical instrument/tool (112, 117, 152, 154, 156, 8016) is finished being utilized (e.g., post procedure). A “dry-run” activation (8066) may include activating an empty end effector (i.e., an end effector not significantly grasping other material) with suitable energy required to sever and/or seal tissue for a suitable amount of time, and then allow end effector to suitably deenergize (e.g., an activation cycle). Of course, “dry-run” activation (8066) may include activating end effector with any suitable level of energy as would be apparent to one skilled in the art in view of the teachings here. Further, “dry-run” activation (8066) may include any suitable type of energized activation (e.g., ultrasonic, RF energy, etc.,) as would be apparent to one skilled in the art in view of the teachings herein.

Surgical instrument/tool (112, 117, 152, 154, 156, 8016) may include any suitable number of sensors configured to measure suitable characteristics during the operation of surgical instrument/tool (112, 117, 152, 154, 156, 8016). Because surgical instrument/tool (112, 117, 152, 154, 156, 8016) is in communication with other suitable components of computer-implemented interactive surgical system (100), such components of system (100) may then collect/measure (8067) data (e.g., temperature, time, and energy output, etc.) during the post procedure activation (8066). While temperature, time, and energy output are measured in the current example, any other suitable variables may be measured as would be apparent to one skilled in the art in view of the teachings herein.

Next, suitable components of system (100) (such as hub (106), generator module (140), etc.) may then compare (8068) data acquired during the dry-run with specified parameters, such as data acquired during the first activation of the actual surgical procedure. If the comparison (8068) verifies that the measured variables from the dry-run activation (8066) are within specified parameters, the assessment method (8064) may correspond to a determination that specific features of surgical instrument/tool (112, 117, 152, 154, 156, 8016) may be recovered, or at least have some capacity for being recovered. Conversely, if the measured variables are not within the specified parameters, the assessment method (8064) may correspond to a determination that specific features of surgical instrument/tool (112, 117, 152, 154, 156, 8016) should be disposed.

Determining the range of specified parameters may be achieved using any suitable means as would be apparent to one skilled in the art in view of the teachings herein. In the current example shown in FIG. 9, system (100) may store and utilize variable measurements taken during a single activation (e.g., the first activation) of surgical instrument/tool (112, 117, 152, 154, 156, 8016) during a surgical procedure and utilized those measurements to generate the range of specified parameters. As another example, system (100) may store and utilize variable measurements taken during multiple activations of surgical instrument/tool (112, 117, 152, 154, 156, 8016) throughout a surgical procedure and utilized those measurements to generate the range of specified parameters. As yet another example, the range of specified parameters may be predetermined.

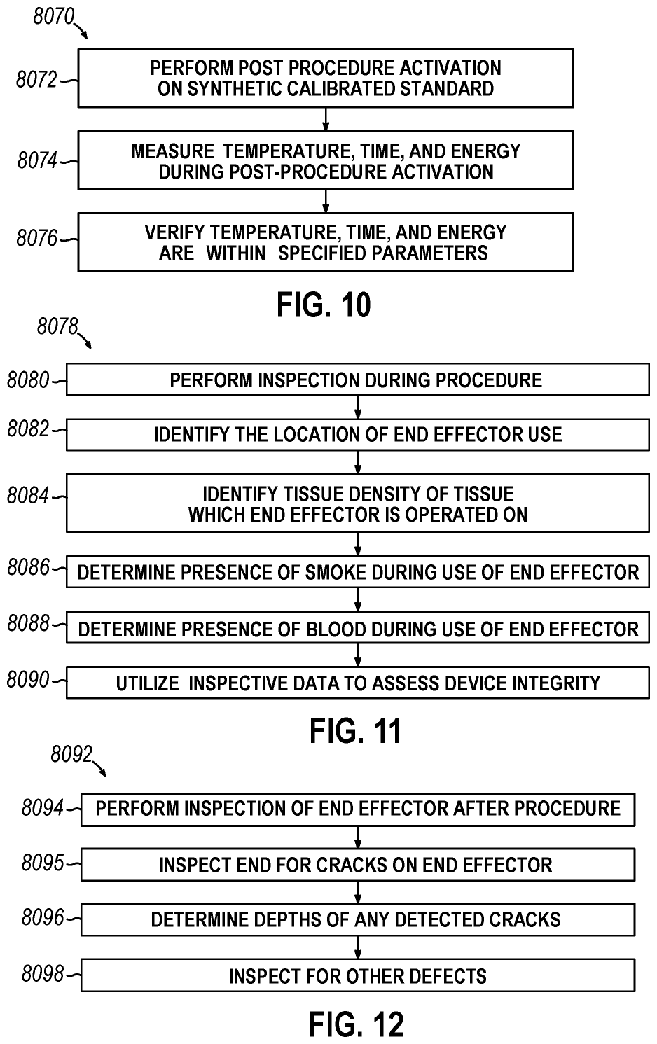

FIG. 10 shows another exemplary assessment method (8070) that may be used to assess the recovery capacity of suitable features of surgical instrument/tool (112, 117, 152, 154, 156, 8016). Therefore, it should be understood that assessment method (8070) may be readily incorporated into method (8050) described above. As will be described below, assessment method (8070) may be substantially similar to assessment method (8064) described above, except that method (8070) utilizes a synthetic calibrated standard (e.g., synthetic tissue) during a post procedure activation (8072) instead of a dry-run.

First, a user may perform a post procedure activation (8072) of surgical instrument/tool (112, 117, 152, 154, 156, 8016) on a piece of synthetic tissue (e.g., a calibrated standard). During post procedure activation (8072), end effector of surgical instrument/tool (112, 117, 152, 154, 156, 8016) may grasp the synthetic tissue and suitably apply energy to synthetic tissue in a similar fashion to exemplary use of end effector with patient tissue during a surgical procedure. Such a piece of synthetic tissue may be provided with surgical instrument/tool (112, 117, 152, 154, 156, 8016) in a surgical kit. Such a piece of synthetic tissue may be a 3D printed “vessel” like structure that surgical instrument/tool (112, 117, 152, 154, 156, 8016) may grasp and “seal” during activation.

Surgical instrument/tool (112, 117, 152, 154, 156, 8016) may include any suitable number of sensors configured to measure suitable characteristics during the operation of surgical instrument/tool (112, 117, 152, 154, 156, 8016). Because surgical instrument/tool (112, 117, 152, 154, 156, 8016) is in communication with other suitable components of computer-implemented interactive surgical system (100), such components of system (100) may then measure (8074) temperature, time, and energy output during the post procedure activation (8072). While temperature, time, and energy output are measured in the current example, any other suitable variables may be measured as would be apparent to one skilled in the art in view of the teachings herein.

Next, suitable components of system (100) (such as hub (106), generator module (140), etc.) may then verify (8076) that the measured variables from the post procedure activation (8072) are within specified parameters, while the user may visually verify end effector suitably applied energy to the synthetic standard (e.g., visually confirm a seal was made). If the measured variables are within the specified parameters and end effector suitably applied energy to the synthetic standard, the assessment method (8070) may correspond to a determination that specific features of surgical instrument/tool (112, 117, 152, 154, 156, 8016) may be recovered, or at least have some capacity for being recovered. Conversely, if the measured variables are not within the specified parameters and/or end effector did not suitably applied energy to the synthetic standard, the assessment method (8070) may correspond to a determination that specific features of surgical instrument/tool (112, 117, 152, 154, 156, 8016) should be disposed.

Determining the range of specified parameters may be achieved using any suitable means as would be apparent to one skilled in the art in view of the teachings herein. In the current example shown in FIG. 10, system (100) may store and utilize variable measurements taken during a single activation (e.g., the first activation) of surgical instrument/tool (112, 117, 152, 154, 156, 8016) during a surgical procedure and utilize those measurements to generate the range of specified parameters. As another example, system (100) may store and utilize variable measurements taken during multiple activations of surgical instrument/tool (112, 117, 152, 154, 156, 8016) throughout a surgical procedure and utilized those measurements to generate the range of specified parameters. As yet another example, the range of specified parameters may be predetermined.

FIG. 11 shows another exemplary assessment method (8078) that may be used to assess the recovery capacity of suitable features of surgical instrument/tool (112, 117, 152, 154, 156, 8016). Therefore, it should be understood that assessment method (8078) may be readily incorporated into method (8050) described above. In the current example, assessment method (8078) may utilize suitable components of surgical visualization system (8010) and/or control system (8020) in order to generate the necessary inspection data described below to determine if features of surgical instrument/tool (112, 117, 152, 154, 156, 8016) have any recovery capacity or should be discarded.

Assessment method (8078) includes utilizing surgical visualization system (8010) to perform inspection (8080) during a surgical procedure. Data acquired during inspection (8080) may be communicated and stored on suitable components of system (100), such as hub (106). During the procedure, various features of visualization system (8010) may be utilized to identify (8082) the anatomical location where end effector of surgical instrument/tool (112, 117, 152, 154, 156, 8016) is being used; and communicate that data to suitable components of system (100). Further, during the procedure, various features of visualization system (8010) may be utilized to identify (8084) the tissue density of tissue which end effector of surgical instrument/tool (112, 117, 152, 154, 156, 8016) is operating on; and communicate that data to suitable components of system (100). Further, during the procedure, various features of visualization system (8010) may be utilized to determine (8086) the presence of smoke during use tissue density of surgical instrument/tool (112, 117, 152, 154, 156, 8016); and communicate that data to suitable components of system (100). Further, during the procedure, various features of visualization system (8010) may be utilized to determine (8088) the presence of blood during use tissue density of surgical instrument/tool (112, 117, 152, 154, 156, 8016); and communicate that data to suitable components of system (100).

Finally, system (100) may utilize (8090) the above acquired inspection data in order to assess the recovery capacity of at least one feature of surgical instrument/tool (112, 117, 152, 154, 156, 8016). If the measured variables are within the specified parameters, the assessment method (8078) may correspond to a determination that specific features of surgical instrument/tool (112, 117, 152, 154, 156, 8016) may be recovered, or at least have some capacity for being recovered. Conversely, if the measured variables are not within the specified parameters, the assessment method (8078) may correspond to a determination that specific features of surgical instrument/tool (112, 117, 152, 154, 156, 8016) should be disposed.

FIG. 12 shows another exemplary assessment method (8092) that may be used to assess the recovery capacity of suitable features of surgical instrument/tool (112, 117, 152, 154, 156, 8016). Therefore, it should be understood that assessment method (8092) may be readily incorporated into method (8050) described above. In the current example, assessment method (8092) may utilize suitable components of surgical visualization system (8010) and/or control system (8020) in order to generate the necessary inspection data described below to determine if features of surgical instrument/tool (112, 117, 152, 154, 156, 8016) have any recovery capacity or should be discarded.

Assessment method (8092) includes utilizing surgical visualization system (8010) to perform inspection (8094) after a surgical procedure. Data acquired during inspection (8092) may be communicated and stored on suitable components of system (100), such as hub (106). After a procedure, various features of visualization system (8010) may be utilized to inspect (8095) end effector of surgical instrument/tool (112, 117, 152, 154, 156, 8016) for cracks; and communicate that data to suitable components of system (100). Further, after a procedure, various features of visualization system (8010) may be utilized to determine (8096) the depths of any crack detected in end effector of surgical instrument/tool (112, 117, 152, 154, 156, 8016) and inspect (8098) for any other defects; and communicate that data to suitable components of system (100).

Finally, system (100) may utilize the above acquired inspection data in order to assess the recovery capacity of at least one feature of surgical instrument/tool (112, 117, 152, 154, 156, 8016). If the measured variables are within the specified parameters, the assessment method (8092) may correspond to a determination that specific features of surgical instrument/tool (112, 117, 152, 154, 156, 8016) may be recovered, or at least have some capacity for being recovered. Conversely, if the measured variables are not within the specified parameters, the assessment method (8092) may correspond to a determination that specific features of surgical instrument/tool (112, 117, 152, 154, 156, 8016) should be disposed.

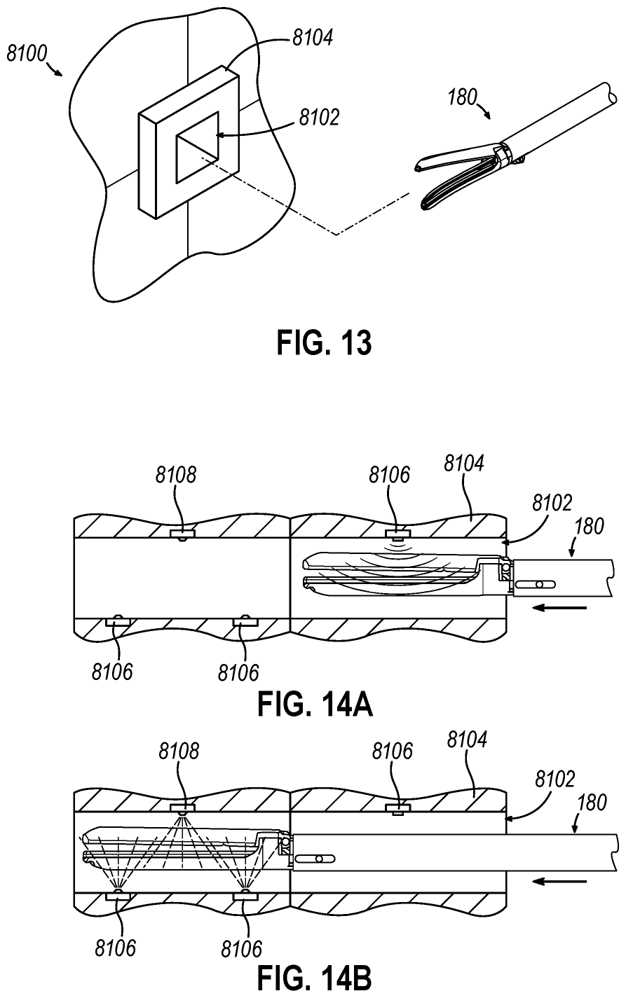

FIGS. 13-14B show an exemplary assessment and cleaning port (8100) that may be readily incorporated into hub (106) and/or a console of visualization system (8010) in order to perform assessment method (8092) described above, and also clean end effector. Assessment and cleaning port (8100) includes a channel (8102) extending from an exterior portal (8104). Exterior portal (8104) may be accessible for an end effector, such as end effector (180) in the current example, to be easily inserted into channel (8102). Likewise, channel (8102) is dimensioned to suitably receive an end effector, such as end effector (180) in the current example.

As shown in FIGS. 14A-14B, an interior surface of port (8100) defining channel (8102) includes at least one visual inspector (8106) and at least one cleaning assembly (8108). In the current example, cleaning assemblies (8108) and visual inspector (8106) are longitudinally offset from each other. However, this is merely optional. During use, end effector (180) may be inserted into channel (8102) such that end effector (180) is adjacent to visual inspector (8106). Visual inspector (8106) may then perform assessment method (8092) described above and determine if features of end effector (180) are recoverable. If recoverable, as shown in FIG. 14B, end effector (180) may be placed adjacent to cleaning assembly (8108) such that cleaning assembly (8108) may apply cleaning materials onto end effector (180). Any suitable cleaning materials may be applied as would be apparent to one skilled in the art in view of the teachings herein.

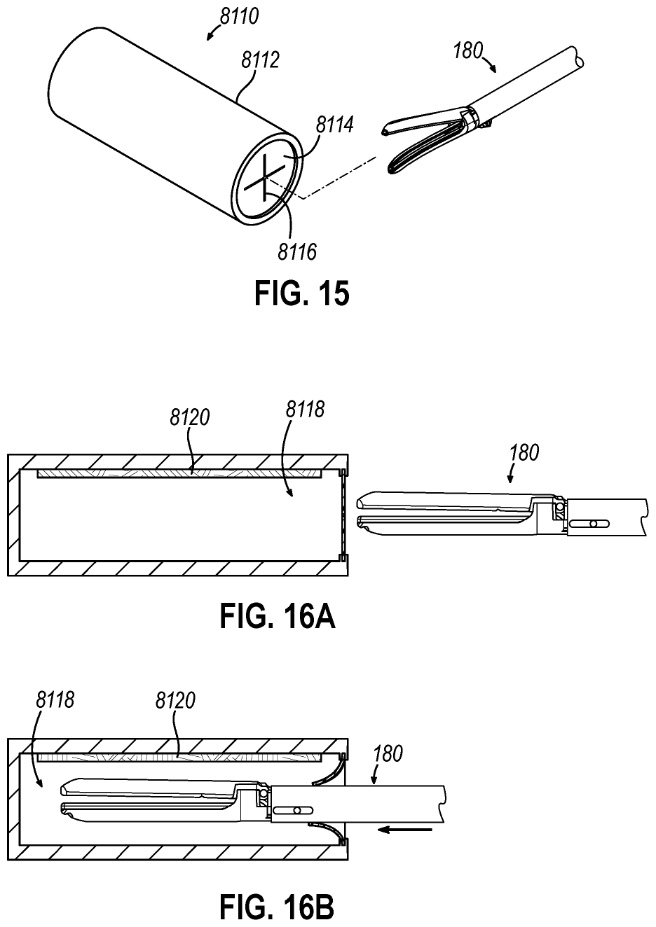

FIGS. 15-16B show an exemplary cleaning sheath (8110) that may be used to cover an end effector, such as end effector (180) shown. As will be described in greater detail below, cleaning sheath (8110) may be applied to end effector (180) after exemplary use in order to prevent other objects, besides end effector (180) and the interior of sheath (8110), from being exposed to biohazard materials accumulated on end effector (180) during exemplary use.

Cleaning sheath (8110) includes a hollow body (8112) defining an interior (8118), a seal (8112) located at an open end of hollow body (8112), and a cleaning material (8120) located within interior (8118) of hollow body (8112). Seal (8112) define an expandable opening (8116) configured to suitably receive end effector (180) such that end effector (180) may be inserted within interior (8118) of hollow body (8112), as shown in FIGS. 16A-16B. In some instances a robotic arm may be configured to attach sheath (8110) with end effector (180).

Seal (8112) may sufficiently engage portions of shaft assembly and/or end effector (180) when suitably inserted into interior (8118) such that seal (8112) inhibits any materials located within interior (8118) from escaping hollow body (8112).

Cleaning material (8120) may include chemicals or other materials that may clean and/or preserve end effector (180) while contained within interior (8118). Cleaning materials (8120) may be configured to clean off debris accumulated on end effector (180) from exemplary use of end effector (180). Cleaning material (8120) may contain any suitable cleaning materials as would be apparent to one skilled in the art in view of the teachings herein. For example, cleaning materials (8120) may include frozen CO2 pellets that may configured to “grit” blast eschar accumulated on end effector (180) from exemplary use.

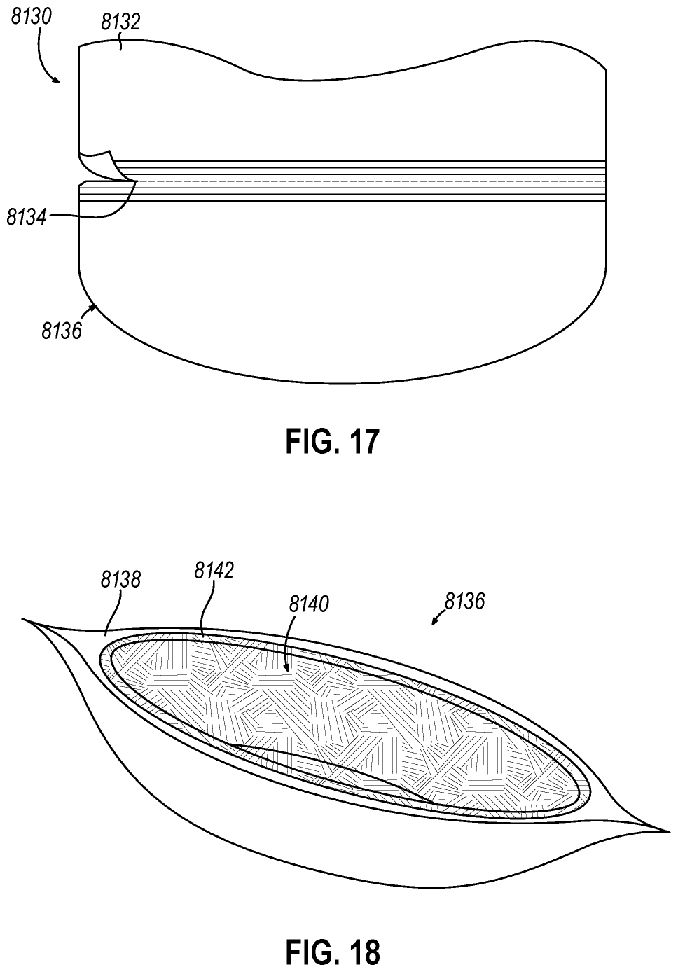

FIG. 17 shows an exemplary surgical instrument packaging (8130) that may be used to transport surgical instrument/tool (112, 117, 152, 154, 156, 8016) into the surgical theater while keeping surgical instrument/tool (112, 117, 152, 154, 156, 8016) suitably sterile. Additionally, as will be described in greater detail below, packaging (8130) includes a removable cleaning kit (8136) that may be utilized in the surgical theater, post procedure or mid-procedure, to clean features of surgical instrument/tool (112, 117, 152, 154, 156, 8016) for purposes of recovery or continued use.

Packaging (8130) includes a primary packaging (8132) configured to store suitable components of a surgical kit used to form surgical instrument/tool (112, 117, 152, 154, 156, 8016). Additionally, packaging (8130) includes removable cleaning kit (8136) that is removably attached to primary packaging (8132) via a perforated section (8134). Therefore, when a user desires to use cleaning kit (8136), they may remove cleaning kit (8136) from primary packaging (8132) via tearing perforated section (8134).

Turning to FIG. 18, in the current example, removable cleaning kit (8136) includes a container (8138) (e.g., a bowl) defining an opening (8140) and a cleaning material (8142) accessible via opening (8140). Cleaning material (8142) may also include a suitable applicator, such as a sponge soaked in a cleaning material. Cleaning material (8142) may include a gritty material or pellets. Cleaning material (8142) may include a bottle brush cleaner, leaflets, or fingers for the user to clean end effector (180) with a cleaning material.

Container (8138) may be moldered into a type of bowl dimensioned to receive liquid cleaning materials (8142). Therefore, container (8138) may act as a basin. The portion of container (8128) forming basin may be lined with a cleaning material that, upon contacting liquid, will self-mix with the liquid. Container (8138) may include a sponge or have a base of sponge molded into it for cleaning purpose. The sponge may be soaked in cleaning fluid to assist in debris removal. The sponge may contain dry chemicals that are activated when becoming wet.

### IV. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A method of determining a recovery capacity of at least one feature of a surgical instrument, the method comprising: (i) establishing communication with the surgical instrument; (ii) assisting operation of the surgical instrument during a procedure; (iii) obtaining data related to the surgical instrument; (iv) evaluating the data obtained related to the surgical instrument to determine a digital assessment of an impact on performance of the at least one feature of the surgical instrument; and (v) determining, based on the digital assessment, a capacity of recovery for the at least one feature of the surgical instrument.

### Example 2

The method of any one or more of the previous Examples, wherein the capacity of recovery relates to the ability of the at least one feature to withstand a procedural aspect required to reuse the at least one feature of the surgical instrument.

### Example 3

The method of any one or more of the previous Examples, wherein the procedural aspect comprises cleaning the at least one feature of the surgical instrument.

### Example 4

The method of any one or more of the previous Examples, wherein the procedural aspect comprises remanufacturing the at least one feature of the surgical instrument.

### Example 5

The method of any one or more of the previous Examples, further comprising providing a recommendation, based on the capacity of recovery.

### Example 6

The method of any one or more of the previous Examples, wherein the recommendation comprises displaying instructions to discard the at least one feature or displaying instructions to recover the at least one feature.

### Example 7

The method of any one or more of the previous Examples, further comprising providing a user feedback option to over-ride the recommendation.

### Example 8

The method of any one or more of the previous Examples, wherein obtaining data occurs concurrently with the procedure.

### Example 9

The method of any one or more of the previous Examples, wherein obtaining data comprises identifying the location, within a patient, of an end effector of the surgical instrument.

### Example 10

The method of any one or more of the previous Examples, wherein obtaining data comprises identifying tissue density which an end effector of the surgical instrument is operating on.

### Example 11

The method of any one or more of the previous Examples, wherein obtaining data comprises defining the presence of smoke or blood during use of an end effector of the surgical instrument.

### Example 12

The method of any one or more of the previous Examples, wherein obtaining data occurs after the procedure.

### Example 13

The method of any one or more of the previous Examples, wherein obtaining data comprises inspecting an end effector of the surgical instrument for cracks.

### Example 14

The method of any one or more of the previous Examples, wherein obtaining data comprises performing a dry-run activation of an end effector of the surgical instrument.

### Example 15

The method of any one or more of the previous Examples, wherein obtaining data comprises performing a test activation of an end effector of the surgical instrument on a synthetic calibration piece.

### Example 16

A surgical system comprising: (a) a surgical instrument comprising an end effector; (b) a hub configured to establish communication with the surgical instrument and assist the surgical instrument during a procedure; (c) an assessment port associated with the hub, wherein the assessment port defines a channel dimensioned to receive the end effector, wherein the assessment port comprises a visualization system located within the channel, wherein the visualization system is configured to inspect the end effector to defects and communicate the results to the hub.

### Example 17

The surgical system of any one or more of the previous Examples, wherein the assessment port further comprises a cleaning assembly located within the channel.

### Example 18

The surgical system of any one or more of the previous Examples, wherein the cleaning assembly is configured to clean the end effector in response to the inspection of the visualization system.

### Example 19

The surgical system of any one or more of the previous Examples, wherein the hub further comprises a generator module configured to power the surgical instrument.

### Example 20

A method of determining a recovery capacity of at least one feature of a surgical instrument, the method comprising: (i) establishing communication with the surgical instrument; (ii) assisting operation of the surgical instrument during a procedure; (iii) obtaining a first set of data about the operation of the surgical instrument during the procedure; (iv) obtaining a second set of data about the operation of the surgical instrument after the procedure; (v) comparing the first set of data with the second set of data to determine a digital assessment of an impact on performance of the at least one feature of the surgical instrument; and (vi) determining, based on the digital assessment, a capacity of recovery for the at least one feature of the surgical instrument.

### V. Miscellaneous

Versions of the devices described above may have application in conventional medical treatments and procedures conducted by a medical professional, as well as application in robotic-assisted medical treatments and procedures.

It should be understood that any of the versions of instruments described herein may include various other features in addition to or in lieu of those described above. By way of example only, any of the instruments described herein may also include one or more of the various features disclosed in any of the various references that are incorporated by reference herein. It should also be understood that the teachings herein may be readily applied to any of the instruments described in any of the other references cited herein, such that the teachings herein may be readily combined with the teachings of any of the references cited herein in numerous ways. Other types of instruments into which the teachings herein may be incorporated will be apparent to those of ordinary skill in the art.

In addition to the foregoing, the teachings herein may be readily combined with the teachings of U.S. Pat. App. No. [ATTORNEY DOCKET NO. END9447USNP1.0754992], entitled “Method of Reclaiming Portions of Surgical Instruments for Remanufacturing and Sustainability,” filed on even date herewith, the disclosure of which is incorporated by reference herein. Various suitable ways in which the teachings herein may be combined with the teachings of U.S. Pat. App. No. [ATTORNEY DOCKET NO. END9447USNP1.0754992] will be apparent to those of ordinary skill in the art in view of the teachings herein.

In addition to the foregoing, the teachings herein may be readily combined with the teachings of U.S. Pat. App. No. [ATTORNEY DOCKET NO. END9448USNP1.0754994], entitled “Surgical Instrument with Predetermined Separation Features for Waste Stream Utilization and Related Methods,” filed on even date herewith, the disclosure of which is incorporated by reference herein. Various suitable ways in which the teachings herein may be combined with the teachings of U.S. Pat. App. No. [ATTORNEY DOCKET NO. END9448USNP1.0754994] will be apparent to those of ordinary skill in the art in view of the teachings herein.

In addition to the foregoing, the teachings herein may be readily combined with the teachings of U.S. Pat. App. No. [ATTORNEY DOCKET NO. END9448USNP2.0754977], entitled “Surgical Instrument with Removable Cable and Associated Couplings,” filed on even date herewith, the disclosure of which is incorporated by reference herein. Various suitable ways in which the teachings herein may be combined with the teachings of U.S. Pat. App. No. [ATTORNEY DOCKET NO. END9448USNP2.0754977] will be apparent to those of ordinary skill in the art in view of the teachings herein.

In addition to the foregoing, the teachings herein may be readily combined with the teachings of U.S. Pat. App. No. [ATTORNEY DOCKET NO. END9448USNP3.0754979], entitled “Surgical System and Methods of Assembly and Disassembly of Surgical Instrument,” filed on even date herewith, the disclosure of which is incorporated by reference herein. Various suitable ways in which the teachings herein may be combined with the teachings of U.S. Pat. App. No. [ATTORNEY DOCKET NO. END9448USNP3.0754979] will be apparent to those of ordinary skill in the art in view of the teachings herein.

In addition to the foregoing, the teachings herein may be readily combined with the teachings of U.S. Pat. App. No. [ATTORNEY DOCKET NO. END9449USNP1.0754981], entitled “Robotic Surgical System with Removable Portion and Method of Disassembling Same,” filed on even date herewith, the disclosure of which is incorporated by reference herein. Various suitable ways in which the teachings herein may be combined with the teachings of U.S. Pat. App. No. [ATTORNEY DOCKET NO. END9449USNP1.0754981] will be apparent to those of ordinary skill in the art in view of the teachings herein.

In addition to the foregoing, the teachings herein may be readily combined with the teachings of U.S. Pat. App. No. [ATTORNEY DOCKET NO. END9450USNP1.0754983], entitled “System for Determining Disposal of Surgical Instrument and Related Methods,” filed on even date herewith, the disclosure of which is incorporated by reference herein. Various suitable ways in which the teachings herein may be combined with the teachings of U.S. Pat. App. No. [ATTORNEY DOCKET NO. END9450USNP1.0754983] will be apparent to those of ordinary skill in the art in view of the teachings herein.

In addition to the foregoing, the teachings herein may be readily combined with the teachings of U.S. Pat. App. No. [ATTORNEY DOCKET NO. END9450USNP2.0754999], entitled “Reclamation Packaging for Surgical Instrument and Related Methods,” filed on even date herewith, the disclosure of which is incorporated by reference herein. Various suitable ways in which the teachings herein may be combined with the teachings of U.S. Pat. App. No. [ATTORNEY DOCKET NO. END9450USNP2.0754999] will be apparent to those of ordinary skill in the art in view of the teachings herein.

In addition to the foregoing, the teachings herein may be readily combined with the teachings of U.S. Pat. App. No. [ATTORNEY DOCKET NO. END9450USNP30.0755001], entitled “Surgical Instrument with Various Alignment Features and Methods for Improved Disassembly and Assembly,” filed on even date herewith, the disclosure of which is incorporated by reference herein. Various suitable ways in which the teachings herein may be combined with the teachings of U.S. Pat. App. No. [ATTORNEY DOCKET NO. END9450USNP3.0755001] will be apparent to those of ordinary skill in the art in view of the teachings herein.

It should also be understood that any ranges of values referred to herein should be read to include the upper and lower boundaries of such ranges. For instance, a range expressed as ranging “between approximately 1.0 inches and approximately 1.5 inches” should be read to include approximately 1.0 inches and approximately 1.5 inches, in addition to including the values between those upper and lower boundaries.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by an operator immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A method of determining a recovery capacity of at least one feature of a surgical instrument, the method comprising:
(i) establishing communication with the surgical instrument;
(ii) assisting operation of the surgical instrument during a procedure;
(iii) obtaining data related to the surgical instrument;
(iv) evaluating the data obtained related to the surgical instrument to determine a digital assessment of an impact on performance of the at least one feature of the surgical instrument; and
(v) determining, based on the digital assessment, a capacity of recovery for the at least one feature of the surgical instrument.

2. The method of claim 1, wherein the capacity of recovery relates to the ability of the at least one feature to withstand a procedural aspect required to reuse the at least one feature of the surgical instrument.

3. The method of claim 2, wherein the procedural aspect comprises cleaning the at least one feature of the surgical instrument.

4. The method of claim 2, wherein the procedural aspect comprises remanufacturing the at least one feature of the surgical instrument.

5. The method of claim 1, further comprising providing a recommendation, based on the capacity of recovery.

6. The method of claim 5, wherein the recommendation comprises displaying instructions to discard the at least one feature or displaying instructions to recover the at least one feature.

7. The method of claim 5, further comprising providing a user feedback option to over-ride the recommendation.

8. The method of claim 1, wherein obtaining data occurs concurrently with the procedure.

9. The method of claim 8, wherein obtaining data comprises identifying the location, within a patient, of an end effector of the surgical instrument.

10. The method of claim 8, wherein obtaining data comprises identifying tissue density which an end effector of the surgical instrument is operating on.

11. The method of claim 8, wherein obtaining data comprises defining the presence of smoke or blood during use of an end effector of the surgical instrument.

12. The method of claim 1, wherein obtaining data occurs after the procedure.

13. The method of claim 12, wherein obtaining data comprises inspecting an end effector of the surgical instrument for cracks.

14. The method of claim 12, wherein obtaining data comprises performing a dry-run activation of an end effector of the surgical instrument.

15. The method of claim 12, wherein obtaining data comprises performing a test activation of an end effector of the surgical instrument on a synthetic calibration piece.

16. A surgical system comprising:
(a) a surgical instrument comprising an end effector;
(b) a hub configured to establish communication with the surgical instrument and assist the surgical instrument during a procedure;
(c) an assessment port associated with the hub, wherein the assessment port defines a channel dimensioned to receive the end effector, wherein the assessment port comprises a visualization system located within the channel, wherein the visualization system is configured to inspect the end effector to defects and communicate the results to the hub.

17. The surgical system of claim 16, wherein the assessment port further comprises a cleaning assembly located within the channel.

18. The surgical system of claim 17, wherein the cleaning assembly is configured to clean the end effector in response to the inspection of the visualization system.

19. The surgical system of claim 16, wherein the hub further comprises a generator module configured to power the surgical instrument.

20. A method of determining a recovery capacity of at least one feature of a surgical instrument, the method comprising:
(i) establishing communication with the surgical instrument;
(ii) assisting operation of the surgical instrument during a procedure;
(iii) obtaining a first set of data about the operation of the surgical instrument during the procedure;
(iv) obtaining a second set of data about the operation of the surgical instrument after the procedure;
(v) comparing the first set of data with the second set of data to determine a digital assessment of an impact on performance of the at least one feature of the surgical instrument; and
(vi) determining, based on the digital assessment, a capacity of recovery for the at least one feature of the surgical instrument.
